(19) United States
(12) Patent Application Publication
(10) Pub. No.: US 2025/0095512 A1
(43) Pub. Date: Mar. 20, 2025

(54) OPERATION TRAINING SYSTEM FOR ULTRASOUND AND OPERATION TRAINING METHOD FOR ULTRASOUND

(71) Applicant: China Medical University, Taichung City (TW)

(72) Inventors: Kai-Sheng Hsieh, Kaohsiung City (TW); Tung-Hua Yang, Taipei City (TW); Yu-Lung Hsu, Taichung City (TW); Shih-Sheng Chang, Taichung City (TW)

(73) Assignee: China Medical University, Taichung City (TW)

(21) Appl. No.: 18/888,567

(22) Filed: Sep. 18, 2024

(30) Foreign Application Priority Data
Sep. 19, 2023 (TW) 112135772
May 10, 2024 (TW) 113117469

(51) Int. Cl. G09B 23/28; G06T 19/00; G09B 5/02

(52) U.S. Cl. CPC G09B 23/28; G06T 19/006; G09B 5/02

(57) ABSTRACT

An operation training system for ultrasound includes a sensing device, a data processing device and a display device. The sensing device is configured to sense a hand movement signal and a voice signal. The data processing device is configured to analyze the hand movement signal, control the virtual hand to move the virtual probe in the virtual scene and perform virtual ultrasound detection, and generate an answer content corresponding to the voice signal based on the question-and-answer model. The display device is configured to display the virtual scene and play the answer content.

### RELATED APPLICATIONS

This application claims priority to Taiwan Application Serial Number 112135772, filed Sep. 19, 2023, and Taiwan Application Serial Number 113117469, filed May 10, 2024, which is herein incorporated by reference.

### BACKGROUND

### Technical Field

The present disclosure relates to an operation training system and an operation training method. More particularly, the present disclosure relates to an operation training system for ultrasound and an operation training method for ultrasound.

### Description of Related Art

Professional ultrasonic operator must undergo strict training to know the organs or parts they want to detect, and to move or rotate the ultrasonic probe appropriately to obtain ultrasound images at a specific position or angle help correctly determine the condition of an organ or part. Furthermore, it is necessary to judge whether an organ or part is normal or abnormal through ultrasound images.

The conventional operation training system for ultrasound using a dummy or a real person to carry out ultrasound detection training. However, in the ultrasound detection of children, it is not easy to make the children lie still on the bed to cooperate with the training, and there are still other problems need to improve.

In view of this, how to develop an operation training system for ultrasound and an operation training method for ultrasound has become a goal that relevant academics/industries want to pursue.

### SUMMARY

According to one aspect of the present disclosure, an operation training system for ultrasound includes a sensing device, a data processing device and a display device. The sensing device is configured to sense a hand of a user to generate a hand movement signal, and capture a speaking voice of the user to generate a voice signal. The data processing device is signally connected to the sensing device. The data processing device includes a display generating module, an analysis module, a recognition module and an interactive module. The display generating module is configured to generate a virtual scene, the virtual scene includes a virtual body, a virtual hand, a virtual probe and a virtual ultrasound monitor. The analysis module is signally connected to the display generating module, the analysis module is configured to analyze the hand movement signal and control the virtual hand to move the virtual probe in the virtual scene and perform virtual ultrasound detection based on the hand movement signal, wherein the display generating module selects one of a plurality of ultrasound data to be played, and switches to another of the plurality of ultrasound data to be played according to a scanning angle of the virtual probe. The recognition module is signally connected to the display generating module and the analysis module, the recognition module is configured to recognize the one of the plurality of ultrasound data to be played to generate a view judgment result and a disease judgment result, and store the view judgment result and the disease judgment result into a question-and-answer model. The interactive module is signally connected to the display generating module and the analysis module, the interactive module is configured to analyze the voice signal to extract at least one keyword, and generate an answer content corresponding to the voice signal based on the question-and-answer model. The display device is signally connected to the display generating module, the display device is configured to display the virtual scene and play the answer content.

According to another aspect of the present disclosure, an operation training method for ultrasound includes performing a virtual scene display step, a sensing step, an ultrasound data displaying step, a recognition step and a question feedback step. The virtual scene display step includes configuring a display generating module of a data processing device of an operation training system for ultrasound to generate a virtual scene, the virtual scene includes a virtual body, a virtual hand, a virtual probe and a virtual ultrasound monitor, the virtual scene is displayed on a display device of the operation training system for ultrasound. The sensing step includes configuring a sensing device to sense a hand of a user to generate a hand movement signal, and capture a speaking voice of the user to generate a voice signal. The ultrasound data displaying step includes configuring an analysis module of the data processing device to analyze the hand movement signal and control the virtual hand to move the virtual probe in the virtual scene and perform virtual ultrasound detection based on the hand movement signal, wherein the display generating module selects one of a plurality of ultrasound data to be played, and switches to another of the plurality of ultrasound data to be played according to a scanning angle of the virtual probe. The recognition step includes configuring a recognition module of the data processing device to recognize the one of the plurality of ultrasound data to be played to generate a view judgment result and a disease judgment result, and store the view judgment result and the disease judgment result into a question-and-answer model. The question feedback step includes configuring an interactive module of the data processing device to analyze the voice signal to extract at least one keyword, and generate an answer content corresponding to the voice signal based on the question-and-answer model, and configuring the display device to display the virtual scene and play the answer content.

### BRIEF DESCRIPTION OF THE DRAWINGS

The present disclosure can be more fully understood by reading the following detailed description of the embodiment, with reference made to the accompanying drawings as follows:

FIG. 1 is a schematic diagram of an operation training system for ultrasound and a user according to the 1st embodiment of the present disclosure.

FIG. 2 is a block diagram of the operation training system for ultrasound according to the FIG. 1.

FIG. 3 is a schematic diagram of a virtual scene of the operation training system for ultrasound according to the FIG. 1.

FIG. 4 is a block flow chart of an operation training method for ultrasound according to the 2nd embodiment of the present disclosure.

FIG. 5 is a flow chart of the operation training method for ultrasound according to the FIG. 4.

FIG. 6 is a block diagram of an operation training system for ultrasound according to the 3rd embodiment of the present disclosure.

FIG. 7 is a block flow chart of an operation training method for ultrasound according to the 4th embodiment of the present disclosure.

### DETAILED DESCRIPTION

The embodiment will be described with the drawings. For clarity, some practical details will be described below. However, it should be noted that the present disclosure should not be limited by the practical details, that is, in some embodiment, the practical details is unnecessary. In addition, for simplifying the drawings, some conventional structures and elements will be simply illustrated, and repeated elements may be represented by the same labels.

In addition, the terms first, second, third, etc. are used herein to describe various elements or components, these elements or components should not be limited by these terms. Consequently, a first element or component discussed below could be termed a second element or component.

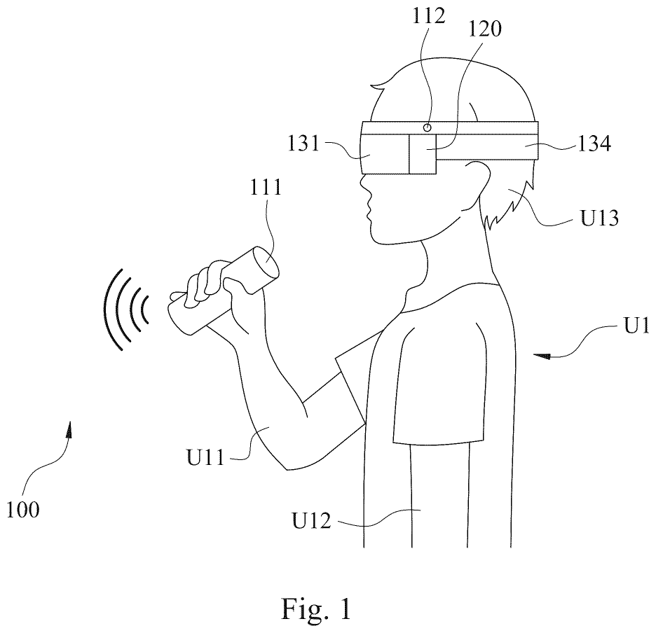

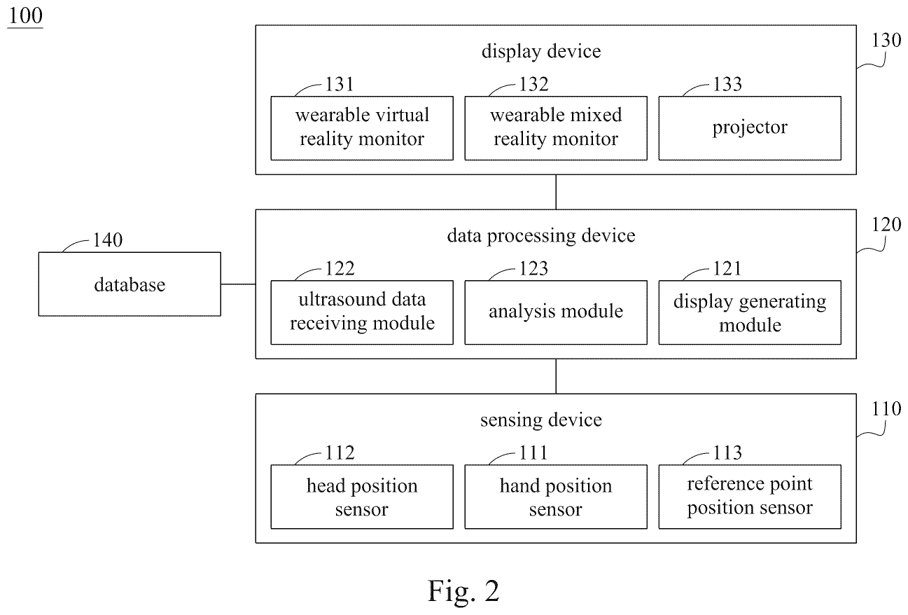

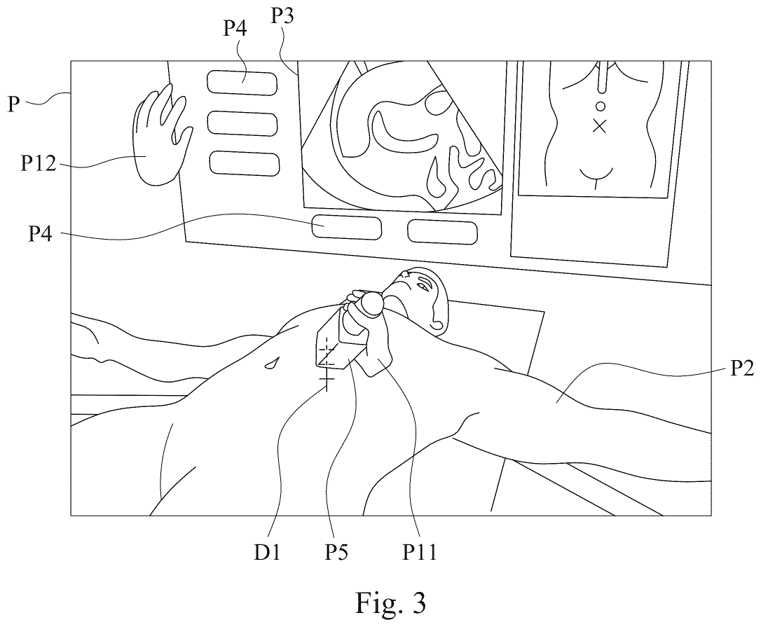

Referring to FIG. 1, FIG. 2 and FIG. 3. FIG. 1 is a schematic diagram of an operation training system 100 for ultrasound and a user U1 according to the 1st embodiment of the present disclosure. FIG. 2 is a block diagram of the operation training system 100 for ultrasound according to the FIG. 1. FIG. 3 is a schematic diagram of a virtual scene P of the operation training system 100 for ultrasound according to the FIG. 1. The operation training system 100 for ultrasound includes a sensing device 110, a data processing device 120 and a display device 130.

The sensing device 110 is configured to sense a hand U11 of a user U1 to generate a hand movement signal. The data processing device 120 is signally connected to the sensing device 110 and includes an analysis module 123 and a display generating module 121. The analysis module 123 is configured to analyze the hand movement signal. The display generating module 121 is signally connected to the analysis module 123 and is configured to generate the virtual scene P, the virtual scene P includes a virtual body P2, a virtual hand P11, a virtual probe P5 and a virtual ultrasound monitor P3. The display device 130 is signally connected to the display generating module 121 and is configured to display the virtual scene P. The virtual hand P11 moves the virtual probe P5 in the virtual scene P and performs virtual ultrasound detection based on the hand movement signal. The analysis module 123 causes the display generating module 121 to select one of a plurality of ultrasound data to be played based on the hand movement signal, and causes the display generating module 121 to display the one of the plurality of ultrasound data to be played on the virtual ultrasound monitor P3. When the analysis module 123 determines that the hand U11 of the user U1 has moved or rotated based on the hand movement signal, it switches to another of the plurality of ultrasound data to be played according to a scanning angle of the virtual probe P5. The display generating module 121 displays another of the plurality of ultrasound data to be played on the virtual ultrasound monitor P3 so as to switch the screen of the virtual ultrasound monitor P3 accordingly.

Thus, the virtual scene P generated by the data processing device 120 can simulate the actual detection scene. The sensing device 110 then senses the hand U11 of the user U1 to allow the user U1 to move the virtual hand P11 in the virtual scene P, and adjust the ultrasound data to be played based on the movement of the hand U11 to increase the authenticity of the simulation training. The details of the operation training system 100 for ultrasound will be described in detail later.

The operation training system 100 for ultrasound can further include a database 140. The database 140 includes at least one ultrasound file. The at least one ultrasound file is divided into the aforementioned plurality of ultrasound data, each of the plurality of ultrasound data is an ultrasound dynamic segment or an ultrasound static frame. The data processing device 120 further includes an ultrasound data receiving module 122, the ultrasound data receiving module 122 is configured to receive the plurality of ultrasound data.

In detail, the data processing device 120 can be an electronic device such as a smart device, a virtual reality processor or a mixed reality processor, etc. The electronic device can include, for example, a central processing unit for calculation and a random access memory generates temporary information along with calculations, and a memory unit such as hard drive. The data processing device 120 can be programmed to form the display generating module 121, the ultrasound data receiving module 122 and the analysis module 123, to execute instructions and corresponding functions. In present embodiment, the data processing device 120 is illustrated as a virtual reality processor. The database 140 can be located in a server or a computer, which can be connected to the data processing device 120 through wireless or wired methods, and can transmit the plurality of ultrasound data to the data processing device 120.

The number of the ultrasound file can be plural, which are dynamic images recorded during the actual operation of ultrasound testing. The ultrasound files can be classified first according to needs, for example, by organs, or by status (normal or abnormal), and can be further classified according to the diseases. In addition, the ultrasound files can be cut according to needs, for example, each complete dynamic image is divided into a plurality of ultrasound dynamic segments and/or ultrasound static frames according to the probe position and/or angle, so each ultrasound files can be divided into a plurality of ultrasound dynamic segments and/or ultrasound static frames. In the present embodiment, the ultrasonic file can be divided by the server and then transmitted to the ultrasound data receiving module 122. In other embodiments, the ultrasonic files can also be transmitted to the data processing device by the server and then divided by the segmentation module of the data processing device, and then transmitted to the ultrasonic data receiving module, but the present disclosure is not limited thereto.

The display device 130 can further include at least one of a wearable virtual reality monitor 131, a wearable mixed reality monitor 132 and a projector 133. Specifically, as shown in FIG. 1, the wearable virtual reality monitor 131 can have a head-mounted display (HMD) structure, so the display device 130 can further include a wearing part 134, which is configured to connect and support the wearable virtual reality monitor 131, and is configured for the user U1 to wear on a head U13. The data processing device 120 can also be located in the wearing part 134. The wearable mixed reality monitor 132 can have a transparent, translucent or see-through near-eye display structure, and can also be connected to another wearing part. In one embodiment, the display device can only include the wearable virtual reality monitor and the projector, so that the virtual scene can be displayed on the wearable virtual reality monitor and the projector at the same time, can facilitate the operator to observe the user's training status.

The sensing device 110 can include a hand position sensor 111, the hand position sensor 111 has a handle structure for the hand U11 to hold. Further, the sensing device 110 can further include a head position sensor 112, the head position sensor 112 can be located in the wearing part 134 to sense the head U13 of the user U1 and send a head movement signal. In addition, the sensing device 110 can further include a reference point position sensor 113.

In detail, the hand position sensor 111 can include an inertial sensing element, a data transmission element, etc. The inertial sensing element senses the movements of the hand U11 (and another hand U12 mentioned later) such as displacement or rotation. Displacement refers to linear displacement such as longitudinal displacement and lateral displacement, and the data transmission element forms the hand movement signal based on the sensing result and transmits it back to the data processing device 120. After analysis by the analysis module 123, the display generating module 121 changes the status of the virtual hand P11 in the virtual scene P based on the hand movement signal. The hand position sensor 111 can further include a tactile feedback module, which can feedback information to the user U1 according to vibration or other methods. In other embodiments, the hand position sensor can have a glove structure for the hand to wear, but the present disclosure is not limited thereto. The structure of the head position sensor 112 is similar to the hand position sensor 111, and can also include inertial sensing elements and data transmission elements to sense the position or the rotation of the head U13 of the user U1 so as to control the change of the angle or position of the virtual scene P.

The reference point position sensor 113 can disposed on a bracket and signally connected to the data processing device 120. Furthermore, it can also signally connected to the hand position sensor 111 and the head position sensor 112 so as to confirm the specific position of each part of the user U1 in the real space, and is convenient for the integration of virtual and real, but the present disclosure is not limited thereto. In other embodiments, the sensing device can include a camera and a plurality of patches. The patches can include two-dimensional codes and can be affixed to the parts of the user's body that are to be sensed and tracked, and the location of each part can be confirmed through image recognition. Alternatively, the sensing device can include an infrared light detector, an infrared light emitter and a reflective patch. The reflective patch is configured to attach on the part of the user's body hat are to be sensed and tracked, and then the infrared light detector and the infrared light transmitter are configured to confirm the position, but the present disclosure is not limited thereto.

Referring to FIG. 1 to FIG. 3. The sensing device 110 can further sense another hand U12 of the user U1, for example, using another hand position sensor 111 to sense the another hand U12 to send another hand movement signal. The virtual scene P generated by the display generating module 121 can further include another virtual hand P12, and the action of the another virtual hand P12 is controlled based on the movement of the another hand U12.

Referring to FIG. 3. The virtual scene P includes the virtual body P2, the virtual hands P11, the another virtual hand P12, the virtual ultrasound monitor P3 and the virtual probe P5. The virtual hands P11, the another virtual hand P12 make displacements or rotations based on the movements of the hand U11 and the another hand U12. The virtual scene P can also include an ultrasound machine, other equipment or instruments in the ward, but the present disclosure is not limited thereto. The virtual scene P can further include at least one button P4, the at least one button P4 is configured to be triggered to change a state of the virtual body in the virtual scene P, freeze a screen of the virtual ultrasound monitor P3, and switch a scan area prompt type on the virtual body P1 or change a scanning theme. In detail, the number of buttons P4 can be plural, and “human body display mode change”, “freeze scanning screen”, “switch scan area”, “heart scan” and “abdominal scan” and other words corresponding to the above functions can be displayed on different buttons P4 respectively. During training, the another virtual hand P12 can be controlled to trigger the button P4 to select perform the heart scan or the abdominal scan, and there is no prompt in the default state of the initial scan. If necessary, the user U1 can control the another virtual hand P12 press the button P4 of “switch display scanning area” to display the prompt of the scanning area on the human body. The prompt can be divided into no prompt, vertical scanning display and horizontal scanning display, and the prompt can be switched by controlling the another virtual hand P12 to press the button P4. In addition, the virtual body P2 can further display a scanning direction D1, which includes a long axis and three short axes. The scan direction D1 is located on the window to be scanned and is available for reference by the user U1. It should be noted that, the ultrasonic scanning in the present disclosure can be applied to the scanning of various organs on the human body, and is not limited to the organs mentioned above.

The user U1 can control the virtual hand P11 to move the virtual probe P5. In the initial state, no data is displayed on the screen of the virtual ultrasound monitor P3. When the user U1 operates the virtual probe P5 to make the angle or the position correct, the virtual ultrasound monitor P3 will display the corresponding ultrasound data. Moreover, when the user U1 continues to move or rotate the hand U11, for example, the movement exceeds a movement threshold, it is determined that the hand U11 has moved. At this time, the ultrasound data displayed by the virtual ultrasound monitor P3 will switch accordingly. That is, switching from the original ultrasound dynamic segment and/or the original ultrasound static frame to another ultrasound dynamic segment and/or another ultrasound static frame. Similarly, when the rotation of the hand U11 of the user U1 exceeds a rotation threshold, it is determined that the hand U11 has rotated. At this time, the ultrasound data displayed by the virtual ultrasound monitor P3 will switch accordingly. The movement threshold can be 15 mm, the rotation threshold can be 10 degrees, and the rotation threshold can be between 8 degrees to 17 degrees, or between 10 degrees to 20 degrees. When the user U1 uses the virtual probe P5 to perform ultrasonic scanning, the user U1 can use the another hand U12 to control the another virtual hand P12, for example, press the button P4 to freeze the scanning screen, but the present disclosure is not limited thereto.

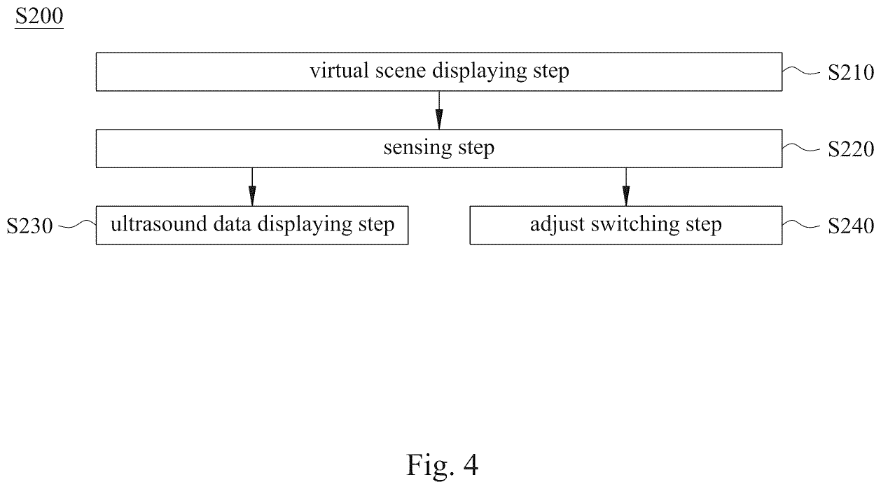

Referring to FIG. 4. FIG. 4 is a block flow chart of an operation training method S200 for ultrasound according to the 2nd embodiment of the present disclosure. The operation training method S200 for ultrasound includes performing a virtual scene displaying step S210, a sensing step S220, an ultrasound data displaying step S230. The details of the operation training method S200 for ultrasound will be described below with the operation training system 100 for ultrasound shown in FIG. 1 to FIG. 3.

In the virtual scene displaying step S210, the display generating module 121 of the data processing device 120 of the operation training system 100 for ultrasound generates the virtual scene P. The virtual scene P includes the virtual body P2, the virtual hand P11, the virtual probe P5 and the virtual ultrasound monitor P3. The virtual scene P is displayed on the display device 130 of the operation training system 100 for ultrasound.

In the sensing step S220, the sensing device 110 senses the hand U11 of the user U1 to generate the hand movement signal.

In the ultrasound data displaying step S230, the analysis module 123 of the data processing device 120 analyzes the hand movement signal, and control the virtual hand P11 to move the virtual probe P5 in the virtual scene P and perform virtual ultrasound detection based on the hand movement signal. The analysis module 123 selects the one of the plurality of ultrasound data to be played based on the hand movement signal, the display generating module 121 displays the one of the plurality of ultrasound data to be played on the virtual ultrasound monitor P3. When the analysis module 123 determines that the hand U11 of the user U1 has moved or rotated based on the hand movement signal, it switches to another of the plurality of ultrasound data to be played according to a scanning angle of the virtual probe P5. The display generating module 121 displays another of the plurality of ultrasound data to be played on the virtual ultrasound monitor P3 so as to switch the screen of the virtual ultrasound monitor P3 accordingly.

In detail, in the virtual scene displaying step S210, the display generating module 121 generates the virtual scene P, and the virtual scene P can be displayed by the wearable virtual reality monitor 131 of the display device 130, allowing the user U1 to perform ultrasound detection training in virtual reality.

In the sensing step S220, the action of the hand U11, such as movement or rotation, is sensed through the hand position sensor 111, so that the virtual hand P11 generates the corresponding response in the virtual scene P action to control the virtual probe P5.

In the ultrasound data displaying step S230, it simulates real ultrasound detection, so that when the virtual probe P5 moves to the corresponding position, the virtual ultrasound monitor P3 in the virtual scene P will display the corresponding ultrasound data, and the virtual ultrasound monitor P3 will switch the screen when the virtual probe P5 moves or rotates. Therefore, the analysis module 123 determines whether the hand U11 of the user U1 has moved or rotated based on the hand movement signal, and can select the corresponding ultrasonic data to be played, and cause the display generating module 121 to switch the screen.

In addition, in the ultrasound data displaying step S230, the sensing device 110 can include a hand position sensor 111, and the hand position sensor 111 includes a handle structure for the hand U11 to hold. When the data processing device 120 determines that the position and angle of the hand U11 are correct based on the hand movement signal, the data processing device 120 activates the hand position sensor 111 generates vibration. That is, at the beginning, there may be no data displayed on the screen of the virtual ultrasound monitor P3, until the user U1 controls the hand U11 to operate the virtual probe P5 makes the angle or the position correct, the virtual ultrasound monitor P3 will display the corresponding ultrasound data. The data processing device 120 can further include a feedback module, the feedback module can issue instructions to activate the tactile feedback module of the hand position sensor 111 to generate vibrations for the user U1 to know the user U1 operates correctly.

Referring to FIG. 4. The operation training method S200 for ultrasound can further include an adjust switching step S240, and the virtual scene P can further include at least one button P4. The user U1 moves the another hand U12, the sensing device 110 can sense the another hand U12 to send another hand movement signal. The analysis module 123 determines whether the another virtual hand P12 in the virtual scene P touches the at least one button P4 based on the another hand movement signal, and configures the display generating module 121 to change a state of the virtual body P2 in the virtual scene P, freeze a screen of the virtual ultrasound monitor P3, and switch a scan area prompt type on the virtual body P2 or change a scanning theme.

In detail, the number of buttons P4 can be plural, therefore, when the another virtual hand P12 in the virtual scene P touches the button P4 of “human body display mode change” to change the human body display mode, a status such as switching the human body skin, seeing through the abdomen, seeing through the heart, etc. of the virtual body P2 in the virtual scene P can be changed. When the another virtual hand P12 in the virtual scene P touches the button P4 of “heart scan” and “abdominal scan” to select perform the heart scan or the abdominal scan, it can switch to the selected scanning theme.

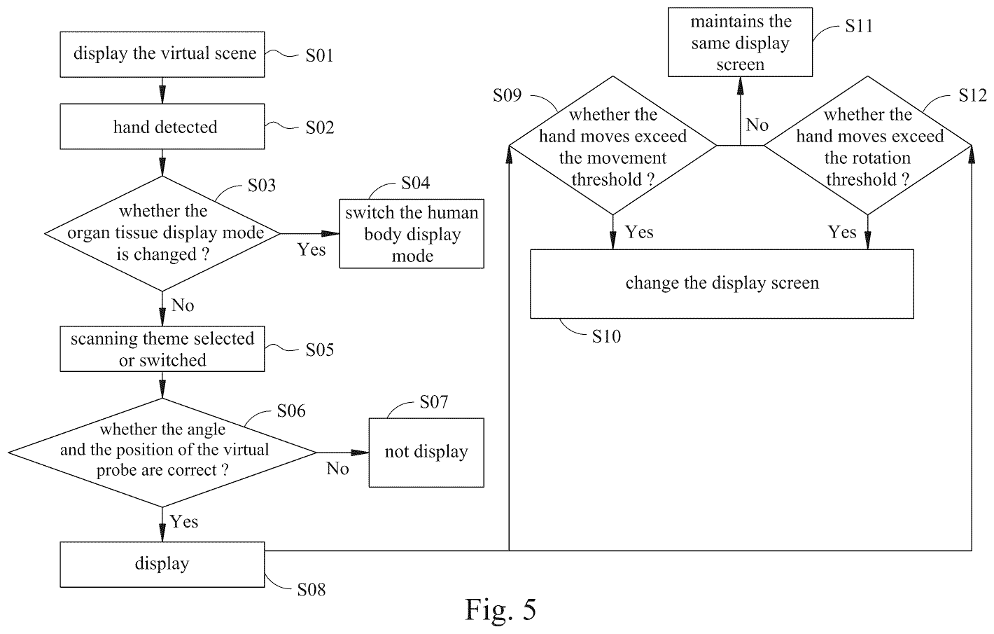

Referring to FIG. 5. FIG. 5 is a flow chart of the operation training method S200 for ultrasound according to the FIG. 4. At the beginning, the user U1 can put on the wearing part 134, place the wearable mixed reality monitor 132 in front of the eyes, and hold the hand position sensor 111 such as a handle structure. In the step S01, the wearable mixed reality monitor 132 of the display device 130 can display the virtual scene P, so that the user U1 can see the virtual body P2, the another virtual hand P11, the another virtual hand P12, the virtual probe P5 and the virtual ultrasound monitor P3 in the virtual scene P.

In the step S02, the hand U11 and the another hand U12 of the user U1 are detected, and the virtual hand P11 and the another virtual hand P12 are moved in the virtual scene P according to the movements of the hands U11 and the another hand U12. In the step S03, confirmed whether the user U1 moves the another hand U12 to make the another virtual hand P12 press the button P4 first, and confirms whether the organ tissue display mode is changed. If the organ tissue display mode is changed (Yes), then enter the step S04 to switch the human body display mode into the human body skin, seeing through the abdomen, seeing through the heart, etc. If the organ tissue display mode is not changed (No), then enter the step S05 to confirm the scanning theme selected or switched by the user U1.

Next, enter the step S06, a correct probe placement position and angle can be preset based on the scanning theme selected or switched by the user U1. Therefore, it can confirm whether the angle and the position of the virtual probe P5 controlled by the user U1 through the virtual hand P11 are correct. If the angle and the position are incorrect (No), then enter the step S07, the display generating module 121 not display the screen. On the other hand, the angle and the position are correct (Yes), then enter the step S08, the display generating module 121 displays the corresponding screen, and the feedback module causes the hand position sensor 111 to vibrate to indicate correct operation. The virtual ultrasound monitor P3 can display the ultrasound dynamic segment for about 2 seconds repeatedly, or a single ultrasound static frame until the screen is switched.

After that, enter the step S09 and the step S12, detect the hand U11 of the user U1 to confirm whether the hand U11 moves exceed the movement threshold (the step S09) or rotates exceed the rotation threshold (the step S12) continuously. If it does not exceed the movement threshold or the rotation threshold (No), then enter the step S11, the display generating module 121 maintains the same display screen on the virtual ultrasound monitor P3. On the other hand (Yes), then enter the step S10 to change the display screen.

It should be noted that, the above-mentioned step flow is only an example, the step S03 and the step S05 can be judged or performed at any time during the operation and are not limited to the above-mentioned order. In addition, when performing the operation training method S200 for ultrasound, the ultrasound data to be displayed can be set in advance. For example, a group of ultrasound data corresponding to the heart and a group corresponding to the abdomen are selected in advance in the database 140. The ultrasonic data corresponding to the heart and the ultrasonic data corresponding to the abdomen are played according to the selection of the user U1, and which ultrasonic dynamic segment and/or ultrasonic static frame of the group corresponding to the heart or abdomen to be played based on the hand U11 of the user U1. However, in other embodiments, a plurality of groups of ultrasound data corresponding to the heart and a plurality of groups of ultrasound data corresponding to the abdomen can also be selected in advance and played randomly, but the present disclosure is not limited thereto. In one embodiment, the data processing device selects “heart scan” or “abdominal scan” according to the user's selection, and requests the database to transmit the corresponding ultrasound data. However, in another embodiment, the data processing device can previously stored at least one group of ultrasound data corresponding to the heart and at least one group of ultrasound data corresponding to the abdomen, but the present disclosure is not limited thereto.

Furthermore, the user U1 can also learn to recognize the positions of various organs by moving the virtual probe P5 in the virtual scene P, and see through the abdomen and see through the heart by switching the human body display mode to make the skin and muscle tissue be transparent so as to identify various organs. Furthermore, it can assist in learning of the scanning position by switching the prompt of the scanning area on the human body.

Furthermore, the operation training method S200 for ultrasound is also used for testing. Therefore, a specific case in the database 140 can be selected as a test subject, that is, the aforementioned ultrasound data is selected from the specific case of the database 140, and can use it as a test topic to take a test. For example, the test topic can be selected by an examiner or artificial intelligence in the electronic device. The test topic can be, for example, organ position, angle, disease symptom confirmation, etc. The examiner can observe the operation of the user U1 through the projector 133 or determine whether the operation is correct. In other embodiments, if the data processing device is a smart device, the virtual scene can also be displayed on the computer screen, but the present disclosure is not limited thereto.

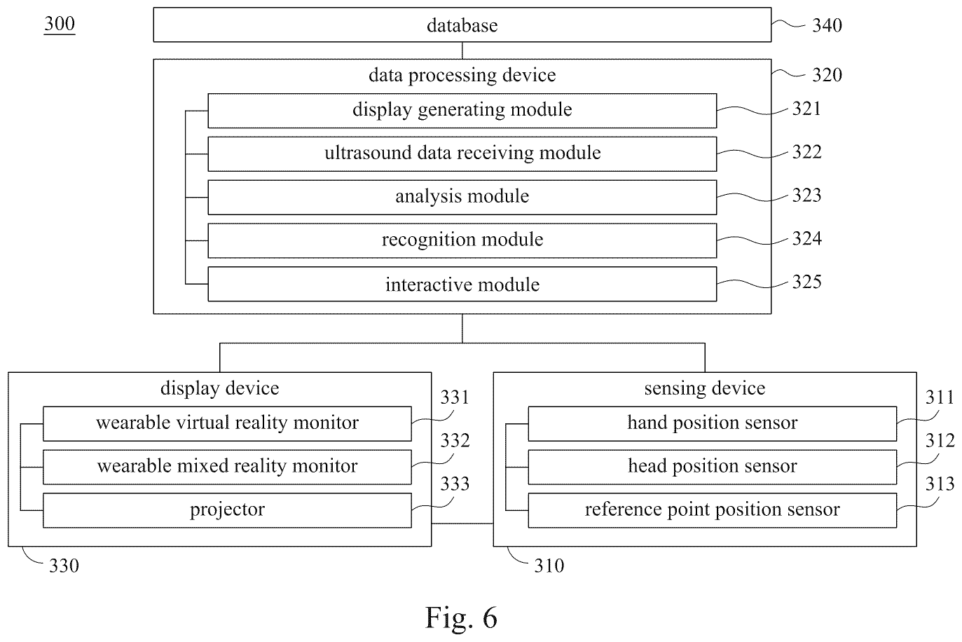

Referring to FIG. 1, FIG. 2 and FIG. 6. FIG. 6 is a block diagram of an operation training system 300 for ultrasound according to the 3rd embodiment of the present disclosure. The operation training system 300 for ultrasound includes a sensing device 310, a data processing device 320, a display device 330 and a database 340. The sensing device 310 includes a hand position sensor 311, a head position sensor 312 and a reference point position sensor 313. The data processing device 320 includes a display generating module 321, an ultrasound data receiving module 322 and an analysis module 323. The display device 330 includes a wearable virtual reality monitor 331, a wearable mixed reality monitor 332, a projector 333 and a wearing part (not shown). In the 3rd embodiment, the hand position sensor 311 and the reference point position sensor 313 of the sensing device 310, the display generating module 321, the ultrasound data receiving module 322 and the analysis module 323 of the data processing device 320, and the display device 330 are the same as the hand position sensor 111 and the reference point position sensor 113 of the sensing device 110, the display generating module 121, the ultrasound data receiving module 122 and the analysis module 123 of the data processing device 120, and the display device 130 of the 1st embodiment respectively, and are not described again herein.

The difference between the 3rd embodiment and the 1st embodiment is that the head position sensor 312 of the sensing device 310 further includes capturing a speaking voice (not shown) of the user U1 to generate a voice signal. The data processing device 320 further includes a recognition module 324 and an interactive module 325. In addition, the database 340 further includes a question-and-answer model (not shown) and a plurality of ultrasound view samples corresponding to different organs and a plurality of ultrasonic disease samples.

The recognition module 324 and the interactive module 325 are signally connected to the display generating module 321 and the analysis module 323. The recognition module 324 is configured to recognize the one of the plurality of ultrasound data to be played to generate a view judgment result and a disease judgment result, and store the view judgment result and the disease judgment result into a question-and-answer model. The recognition module 324 extracts at least one feature of the one of the plurality of ultrasound data to be played, compares the at least one feature with the ultrasound view samples of the database 340 to generate the view judgment result, and compares the at least one feature with the ultrasound disease samples of the database 340 to generate the disease judgment result.

In detail, referring to FIG. 1, FIG. 3 and FIG. 6. When the user U1 scans various organs of the virtual body P2, the recognition module 324 can automatically recognize the image of the one of the plurality of ultrasound data to be played on the virtual ultrasound monitor P3 through the convolutional neural network (CNN), and confirm the view of the one of the plurality of ultrasound data to be played, and confirm whether there is any disease in the organ at the same time.

The interactive module 325 is configured to analyze the voice signal to extract at least one keyword, and generate an answer content corresponding to the voice signal based on the question-and-answer model. The question-and-answer model receives the at least one keyword and operates to generate the answer content based on the at least one keyword. In detail, the user U1 uses the speaking voice to ask questions about the ultrasound operation to the interactive module 325. The interactive module 325 can extract keywords from the voice signal of the speaking voice, the question-and-answer model searches the question-and-answer collection through artificial intelligence (AI) to generate answer content, and automatically displays the answer content in voice through the display device 330 to reply the user U1. Hence, in the role of a virtual teacher to interact with the user U1 and serve as a guide or reminder when the user U1 performs simulated operations. In addition, in other possible embodiments, the user can control the display generating module to correct the angle of the virtual probe or control the buttons in the virtual scene through the voice, but the present disclosure is not limited thereto.

For example, when the user U1 is scanning the heart organ of the virtual body P2, the recognition module 324 will identify the plurality of ultrasound data to be played first, and generate the view judgment result of “parasternal long axis view” and the disease judgment result of “no abnormality”, are store the view judgment result and the disease judgment result to the question-and-answer model. When the user U1 ask “What is the view corresponding to the currently heart scanning?” by speaking, the interactive module 325 can correspondingly retrieve the keywords “heart” and “view”, and the question-and-answer model generate the answer content of “The view corresponding to the currently heart scanning is parasternal long axis view” through the keywords and the aforementioned view judgment result, and automatically reply the answer content to the user U1 by voice through the display device 330.

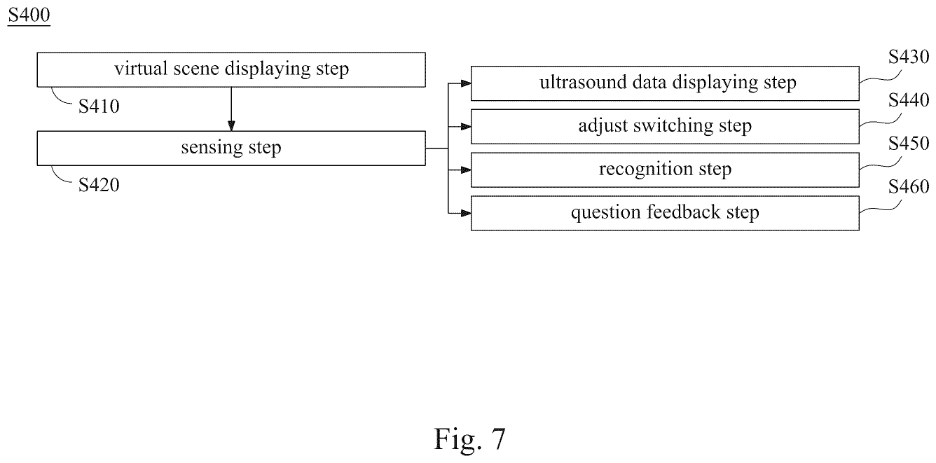

Referring to FIG. 1, FIG. 6 and FIG. 7. FIG. 7 is a block flow chart of an operation training method S400 for ultrasound according to the 4th embodiment of the present disclosure. The operation training system 300 for ultrasound is configured to implement the operation training method S400 for ultrasound, it should be noted that, the operation training method S400 for ultrasound of the present disclosure is not limited to be implemented by the operation training system 300 for ultrasound of the 3rd embodiment of the present disclosure. In the 4th embodiment, the operation training method S400 for ultrasound includes performing a virtual scene displaying step S410, a sensing step S420, an ultrasound data displaying step S430, an adjust switching step S440, a recognition step S450 and a question feedback step S460. The virtual scene displaying step S410, the ultrasound data displaying step S430 and the adjust switching step S440 are the same as the virtual scene displaying step S210, the ultrasound data displaying step S230 and the adjust switching step S240 of the 2nd embodiment respectively, and are not described again herein.

In the sensing step S420, the sensing device 310 senses the hand U11 of the user U1 to generate the hand movement signal, and captures the speaking voice (not shown) of the user U1 to generate a voice signal. In the recognition step S450, the recognition module 324 recognizes the one of the plurality of ultrasound data to be played to generate the view judgment result and the disease judgment result, and store the view judgment result and the disease judgment result into a question-and-answer model. The recognition module 324 extracts the at least one feature of the one of the plurality of ultrasound data to be played, and compares the at least one feature with the plurality of ultrasound view samples of the database 340 to generate the view judgment result, and compares the at least one feature with a plurality of ultrasound disease samples of the database 340 to generate the disease judgment result.

In the question feedback step S460, the interactive module 325 analyzes the voice signal to extract the at least one keyword. The interactive module 325 generates the answer content corresponding to the voice signal based on the question-and-answer model by receiving the at least one keyword. The display device 330 displays the virtual scene P and plays the answer content.

In view of the above, the present disclosure has the following advantages. First, the virtual scene generated by the data processing device can simulate the actual detection scene, and the user's hand is sensed by the sensing device to allow the user to move the virtual hand in the virtual scene, and can adjust the ultrasound data to be played according to the movement of the hand, which can increase the authenticity of the simulation training. Second, through the interactive module as the role of the virtual teacher to interact with the user and serve as a guide or reminder when the user performs simulated operations, which can improve the effect of simulation training.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An operation training system for ultrasound, comprising:
a sensing device configured to sense a hand of a user to generate a hand movement signal, and capture a speaking voice of the user to generate a voice signal;
a data processing device signally connected to the sensing device and comprising:
a display generating module configured to generate a virtual scene, the virtual scene comprising a virtual body, a virtual hand, a virtual probe and a virtual ultrasound monitor;
an analysis module signally connected to the display generating module, the analysis module configured to analyze the hand movement signal and control the virtual hand to move the virtual probe in the virtual scene and perform virtual ultrasound detection based on the hand movement signal, wherein the display generating module selects one of a plurality of ultrasound data to be played, and switches to another of the plurality of ultrasound data to be played according to a scanning angle of the virtual probe;
a recognition module signally connected to the display generating module and the analysis module, the recognition module configured to recognize the one of the plurality of ultrasound data to be played to generate a view judgment result and a disease judgment result, and store the view judgment result and the disease judgment result into a question-and-answer model; and
an interactive module signally connected to the display generating module and the analysis module, the interactive module configured to analyze the voice signal to extract at least one keyword, and generate an answer content corresponding to the voice signal based on the question-and-answer model; and
a display device signally connected to the display generating module, the display device configured to display the virtual scene and play the answer content.

2. The operation training system for ultrasound according to claim 1, further comprising:
a database comprising a plurality of ultrasound view samples and a plurality of ultrasound disease samples corresponding to different organs;
wherein the recognition module extracts at least one feature of the one of the plurality of ultrasound data to be played, compares the at least one feature with the ultrasound view samples to generate the view judgment result, and compares the at least one feature with the ultrasound disease samples to generate the disease judgment result.

3. The operation training system for ultrasound according to claim 2, wherein the database further comprises the question-and-answer model, the question-and-answer model receives the at least one keyword and operates to generate the answer content based on the at least one keyword.

4. The operation training system for ultrasound according to claim 1, further comprising:
a database comprising at least one ultrasound file, the at least one ultrasound file being divided into the plurality of ultrasound data, each of the plurality of ultrasound data being an ultrasound dynamic segment or an ultrasound static frame, and the data processing device further comprising an ultrasound data receiving module configured to receive the plurality of ultrasound data.

5. The operation training system for ultrasound according to claim 1, wherein the display device further comprises a wearing part, the wearing part is configured to connect and support a wearable virtual reality monitor, and is configured for the user to wear on a head; and
the sensing device further comprises a head position sensor, the head position sensor is located in the wearing part to sense the head of the user and send a head movement signal and the voice signal.

6. The operation training system for ultrasound according to claim 1, wherein the virtual scene further comprises at least one button, the at least one button is configured to be triggered to change a state of the virtual body in the virtual scene, freeze a screen of the virtual ultrasound monitor, and switch a scan area prompt type on the virtual body or change a scanning theme.

7. An operation training method for ultrasound, comprising:
performing a virtual scene display step, wherein the virtual scene display step comprises configuring a display generating module of a data processing device of an operation training system for ultrasound to generate a virtual scene, the virtual scene comprises a virtual body, a virtual hand, a virtual probe and a virtual ultrasound monitor, the virtual scene is displayed on a display device of the operation training system for ultrasound;
performing a sensing step, wherein the sensing step comprises configuring a sensing device to sense a hand of a user to generate a hand movement signal, and capture a speaking voice of the user to generate a voice signal;
performing an ultrasound data displaying step, wherein the ultrasound data displaying step comprises configuring an analysis module of the data processing device to analyze the hand movement signal and control the virtual hand to move the virtual probe in the virtual scene and perform virtual ultrasound detection based on the hand movement signal, wherein the display generating module selects one of a plurality of ultrasound data to be played, and switches to another of the plurality of ultrasound data to be played according to a scanning angle of the virtual probe;
performing a recognition step, wherein the recognition step comprises configuring a recognition module of the data processing device to recognize the one of the plurality of ultrasound data to be played to generate a view judgment result and a disease judgment result, and store the view judgment result and the disease judgment result into a question-and-answer model; and
performing a question feedback step, wherein the question feedback step comprises configuring an interactive module of the data processing device to analyze the voice signal to extract at least one keyword, and generate an answer content corresponding to the voice signal based on the question-and-answer model, and configuring the display device to display the virtual scene and play the answer content.

8. The operation training method for ultrasound according to claim 7, further comprising:
performing an adjust switching step, wherein the virtual scene further displays at least one button, the user moves another hand, and the sensing device senses the another hand to send another hand movement signal, wherein the adjust switching step comprises configuring the analysis module to determine whether another virtual hand in the virtual scene touches the at least one button based on the another hand movement signal, and configuring the display generating module to change a state of the virtual body in the virtual scene, freeze a screen of the virtual ultrasound monitor, and switch a scan area prompt type on the virtual body or change a scanning theme.

9. The operation training method for ultrasound according to claim 7, wherein the recognition step further comprises configuring the recognition module to extract at least one feature of the one of the plurality of ultrasound data to be played, and compare the at least one feature with a plurality of ultrasound view samples of a database to generate the view judgment result, and compare the at least one feature with a plurality of ultrasound disease samples of the database to generate the disease judgment result;
wherein the ultrasound view samples and the ultrasound disease samples of the database corresponding to different organs.

10. The operation training method for ultrasound according to claim 7, wherein the question feedback step further comprises configuring the question-and-answer model to receive the at least one keyword and operate to generate the answer content based on the at least one keyword.

11. The operation training method for ultrasound according to claim 9, wherein the database comprises at least one ultrasound file, the at least one ultrasound file is divided into the plurality of ultrasound data, each of the plurality of ultrasound data is an ultrasound dynamic segment or an ultrasound static frame, and the data processing device further comprises an ultrasound data receiving module configured to receive the plurality of ultrasound data.

12. The operation training method for ultrasound according to claim 7, wherein the display device further comprises a wearing part, the wearing part is configured to connect and support a wearable virtual reality monitor, and is configured for the user to wear on a head; and
the sensing device further comprises a head position sensor, the head position sensor is located in the wearing part to sense the head of the user and send a head movement signal and the voice signal.
